(19) United States
(12) Patent Application Publication
(10) Pub. No.: US 2002/0076770 A1
(43) Pub. Date: Jun. 20, 2002

(54) Process for the fermentative preparation of D-pantothenic acid using Coryneform bacteria

(75) Inventors: Nicole Dusch, Werther (DE); Georg Thierbach, Bielefeld (DE)

Correspondence Address: OBLON SPIVAK MCCLELLAND MAIER & NEUSTADT PC, FOURTH FLOOR, 1755 JEFFERSON DAVIS HIGHWAY, ARLINGTON, VA, 22202, US

(73) Assignee: DEGUSSA AG, Duesseldorf (DE)

(21) Appl. No.: 09/953,260

(22) Filed: Sep. 17, 2001

(30) Foreign Application Priority Data
Sep. 23, 2000 (DE) 100 47 142.0

(51) Int. Cl. C12P 13/04; C12N 1/21

(52) U.S. Cl. 435/106; 435/252.3

(57) ABSTRACT

The invention relates to a process for preparing D-pantothenic acid using Coryneform bacteria in which the zwa1 gene is enhanced.

### CROSS-REFERENCE TO RELATED APPLICATION

The present application claims priority to German Application No. DE10047142.0 filed Sep. 23, 2000, the entire contents of which are incorporated herein by reference

### BACKGROUND OF THE INVENTION

1. Field of the Invention

The invention relates to a process for preparing D-pantothenic acid using Coryneform bacteria in which the zwa1 gene is enhanced.

2. Discussion of the Background

Pantothenic acid is a vitamin of commercial importance, which is used in cosmetics, human medicine, the pharmaceuticals industry, human nutrition and in animal nutrition.

Pantothenic acid can be prepared by chemical synthesis, or biotechnologically by fermentation of suitable microorganisms in suitable nutrient solutions. In the chemical synthesis, DL-pantolactone is an important intermediate stage. It is prepared in a multi-stage process from formaldehyde, isobutylaldehyde and cyanide. In further process steps, the racemic mixture is separated, D-pantolactone is subjected to a condensation reaction with β-alanine, and the desired D-pantothenic acid is obtained in this way.

The advantage of the fermentative preparation by microorganisms lies in the direct formation of the desired stereoisomeric D-form, which is free from L-pantothenic acid.

Various types of bacteria, such as e.g. Escherichia coli, Arthrobacter ureafaciens, Corynebacterium erythrogenes, Brevibacterium ammoniagenes, and also yeasts, such as e.g. Debaromyces castellii, can produce D-pantothenic acid in a nutrient solution which comprises glucose, DL-pantoic acid and β-alanine, as shown in EP-A 0 493 060. EP-A 0 493 060 shows that in the case of Escherichia coli (E. coli), the formation of D-pantothenic acid is improved by amplification of pantothenic acid biosynthesis genes from E. coli which are contained on the plasmids pFV3 and pFV5 in a nutrient solution comprising glucose, DL-pantoic acid and β-alanine.

EP-A 0 590 857 and U.S. Pat. No. 5,518,906 describe mutants derived from Escherichia coli strain IFO3547, such as FV5714, FV525, FV814, FV521, FV221, FV6051 and FV5069, which carry resistances to various antimetabolites, such as salicylic acid, α-ketobutyric acid, β-hydroxyaspartic acid, O-methylthreonine and α-ketoisovaleric acid. They produce pantoic acid in a nutrient solution comprising glucose, and D-pantothenic acid in a nutrient solution comprising glucose and β-alanine. Furthermore, EP-A 0 590 857 and U.S. Pat. No. 5,518,906 show that after amplification of the pantothenic acid biosynthesis genes contained on the plasmid pFV31, in the above-mentioned strains the production of D-pantoic acid in nutrient solutions comprising glucose and the production of D-pantothenic acid in a nutrient solution comprising glucose and β-alanine is improved. Processes for the preparation of D-pantothenic acid with the aid of Corynebacterium glutamicum are described only in some instances in the literature. Sahm and Eggeling (Applied and Environmental Microbiology 65(5), 1973-1979, (1999)) thus report on the influence of over-expression of the panB and panC genes and Dusch et al. (Applied and Environmental Microbiology 65(4), 1530-1539, (1999)) report on the influence of the panD gene on the formation of D-pantothenic acid.

However, there remains a need for improved methods of producing pantothenic acid in Coryneform bacteria. On a commercial or industrial scale even small improvements in the yield of pantothenic acid, or the efficiency of their production, are economically significant. Prior to the present invention, it was not recognized that enhancement of the zwa1 gene in Coryneform bacteria would improve pantothenic acid yields.

### SUMMARY OF THE INVENTION

One object of the present invention, is providing a new process for producing D-pantothenic acid by culturing a Coryneform bacteria comprising an enhanced zwa1 gene and collecting the D-pantothenic acid produced. In preferred embodiments of the invention, the zwa1 gene encodes a polypeptide comprising the amino acid sequence of SEQ ID NO:2 or which zwa1 gene comprise SEQ ID NO:1. In another embodiment, the zwa1 gene comprises those nucleotide sequences which hybridize under stringent conditions to SEQ ID NO:1 and encode a polypeptide having Zwa1 protein activity where the stringent conditions comprise washing in 5×SSC at a temperature of from 50 to 68° C.

Another object of the present invention is to prepare D-pantothenic acid having in bacteria with enhanced zwa1 gene and also having enhanced expression of one or more of panB, panC, and/or ilvD.

In one embodiment the zwa1 gene is enhanced by overexpression.

The above objects highlight certain aspects of the invention. Additional objects, aspects and embodiments of the invention are found in the following detailed description of the invention.

### BRIEF DESCRIPTION OF THE DRAWINGS

FIG. 1: Map of the plasmid pCR2.1zwa1exp.

FIG. 2: Map of the plasmid pEC-T18mob2zwa1exp.

FIG. 3: Map of the plasmid pEC-T18mob2.

### DETAILED DESCRIPTION OF THE INVENTION

Unless otherwise defined, all technical and scientific terms used herein have the same meaning as commonly understood by one of ordinary skill in the art of molecular biology. Although methods and materials similar or equivalent to those described herein can be used in the practice or testing of the present invention, suitable methods and materials are described herein. All publications, patent applications, patents, and other references mentioned herein are incorporated by reference in their entirety. In addition, the materials, methods, and examples are illustrative only and are not intended to be limiting.

Reference is made to standard textbooks of molecular biology that contain definitions and methods and means for carrying out basic techniques, encompassed by the present invention. See, for example, Maniatis et al., Molecular Cloning: A Laboratory Manual, Cold Spring Harbor Laboratory, New York (1982) and Sambrook et al., Molecular Cloning: A Laboratory Manual, Cold Spring Harbor Laboratory, New York (1989) and the various references cited therein.

“D-pantothenic acid” or “pantothenic acid” or “pantothenate” as used herein mean the free acids and the salts of D-pantothenic acid, such as calcium, sodium, ammonium or potassium salts.

The invention provides a process for the fermentative preparation of D-pantothenic acid using Coryneform bacteria in which at least the nucleotide sequence which codes for the zwa1 gene product (zwa1 gene) is enhanced, in particular over-expressed.

The strains employed optionally already produce D-pantothenic acid before enhancement of the zwa1 gene.

The term “enhancement” in this connection describes the increase in the intracellular activity of one or more enzymes (proteins) in a microorganism which are coded by the corresponding DNA, for example by increasing the number of copies of the gene or genes, using a potent promoter or using a gene which codes for a corresponding enzyme (protein) having a high activity, and optionally combining these measures.

By enhancement measures, in particular over-expression, the activity or concentration of the corresponding protein is in general increased by at least 10%, 25%, 50%, 75%, 100%, 150%, 200%, 300%, 400% or 500%, up to a maximum of 1000% or 2000%, based on the starting microorganism.

Preferably, a Coryneform bateria with enhanced expression of the zwa1 gene will improve pantothenic acid productivity at least 1% compared to a bacteria which does not contain such an enhanced zwa1 gene.

The microorganisms which the present invention provides can produce D-pantothenic acid from glucose, sucrose, lactose, fructose, maltose, molasses, starch, cellulose or from glycerol and ethanol. They are representatives of Coryneform bacteria, in particular of the genus Corynebacterium. Of the genus Corynebacterium, there may be mentioned in particular the species Corynebacterium glutamicum (C. glutamicum), which is known among experts for its ability to produce L-amino acids.

Suitable strains of the genus Corynebacterium, in particular of the species Corynebacterium glutamicum, are, for example, the known wild-type strains

Corynebacterium glutamicum ATCC13032

Corynebacterium acetoglutamicum ATCC15806

Corynebacterium acetoacidophilum ATCC13870

Corynebacterium thermoaminogenes FERM BP-1539

Brevibacterium flavum ATCC14067

Brevibacterium lactofermentum ATCC13869 and

Brevibacterium divaricatum ATCC14020

and D-pantothenic acid-producing mutants prepared therefrom, such as, for example

Corynebacterium glutamicum ATCC13032 ilvA/pEC7panBC

Corynebacterium glutamicum ATCC13032/pND-D2

It has been found that Coryneform bacteria produce pantothenic acid in an improved manner after over-expression of the zwa1 gene which codes for the Zwa1 gene product.

The nucleotide sequence of the zwa1 gene is shown in SEQ ID No 1 and the enzyme protein amino acid sequence resulting therefrom is shown in SEQ ID No 2.

The zwa1 gene described in SEQ ID No 1 can be employed according to the invention. Alleles of the zwa1 gene which result from the degeneracy of the genetic code or due to “sense mutations” of neutral function can furthermore be used. The polynucleotides of the invention include a polynucleotide according to SEQ ID No. 1 or a fragment prepared therefrom, and also polynucleotides that are at least especially from 70% to 80%, preferably at least from 81% to 85%, especially preferably at least from 86% to 90%, and very especially preferably at least 91%, 93%, 95%, 97% or 99%, identical with the polynucleotide according to SEQ ID No. 1, or with a fragment prepared therefrom.

Homology, sequence similarity or sequence identity of nucleotide or amino acid sequences may be determined conventionally by using known software or computer programs such as the BestFit or Gap pairwise comparison programs (GCG Wisconsin Package, Genetics Computer Group, 575 Science Drive, Madison, Wis. 53711). BestFit uses the local homology algorithm of Smith and Waterman, Advances in Applied Mathematics 2: 482-489 (1981), to find the best segment of identity or similarity between two sequences. Gap performs global alignments: all of one sequence with all of another similar sequence using the method of Needleman and Wunsch, J. Mol. Biol. 48:443-453 (1970). When using a sequence alignment program such as BestFit, to determine the degree of sequence homology, similarity or identity, the default setting may be used, or an appropriate scoring matrix may be selected to optimize identity, similarity or homology scores. Similarly, when using a program such as BestFit to determine sequence identity, similarity or homology between two different amino acid sequences, the default settings may be used, or an appropriate scoring matrix, such as blosum45 or blosum80, may be selected to optimize identity, similarity or homology scores.

Similarly polynucleotides which hybridize under stringent conditions to the zwa1 gene described in SEQ ID No 1 and which have the activity of the Zwa1 protein can be employed according to the invention.

The terms “stringent conditions” or “stringent hybridization conditions” includes reference to conditions under which a polynucleotide will hybridize to its target sequence, to a detectably greater degree than other sequences (e.g., at least 2-fold over background). Stringent conditions are sequence-dependent and will be different in different circumstances. By controlling the stringency of the hybridization and/or washing conditions, target sequences can be identified which are 100% complementary to the probe (homologous probing). Alternatively, stringency conditions can be adjusted to allow some mismatching in sequences so that lower degrees of similarity are detected (heterologous probing).

Typically, stringent conditions will be those in which the salt concentration is less than about 1.5 M Na ion, typically about 0.01 to 1.0 M Na ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. Exemplary low stringency conditions include hybridization with a buffer solution of 30 to 35% formamide, 1 M NaCl, 1% SDS (sodium dodecyl sulphate) at 37° C., and a wash in 1× to 2×SSC (20×SSC=3.0 M NaCl/0.3 M trisodium citrate) at 50 to 55° C. Exemplary moderate stringency conditions include hybridization in 40 to 45% formamide, 1 M NaCl, 1% SDS at 37° C., and a wash in 0.5×to 1×SSC at 55 to 60° C. Exemplary high stringency conditions include hybridization in 50% formamide, 1 M NaCl, 1% SDS at 37° C., and a wash in 0.1×SSC at 60 to 65° C.

Specificity is typically the function of post-hybridization washes, the critical factors being the ionic strength and temperature of the final wash solution. For DNA—DNA hybrids, the Tm can be approximated from the equation of Meinkoth and Wahl, Anal. Biochem., 138:267-284 (1984): Tₘ=81.5° C.+16.6 (log M)+0.41 (% GC)-0.61 (% form)-500/L; where M is the molarity of monovalent cations, % GC is the percentage of guanosine and cytosine nucleotides in the DNA, % form is the percentage of formamide in the hybridization solution, and L is the length of the hybrid in base pairs. The Tₘ is the temperature (under defined ionic strength and pH) at which 50% of a complementary target sequence hybridizes to a perfectly matched probe. Tₘ is reduced by about 1° C. for each 1% of mismatching; thus, Tₘ, hybridization and/or wash conditions can be adjusted to hybridize to sequences of the desired identity. For example, if sequences with approximately 90% identity are sought, the Tₘ can be decreased 10° C. Generally, stringent conditions are selected to be about 5° C. lower than the thermal melting point (Tₘ) for the specific sequence and its complement at a defined ionic strength and pH. However, severely stringent conditions can utilize a hybridization and/or wash at 1, 2, 3, or 4° C. lower than the thermal melting point (Tₘ); moderately stringent conditions can utilize a hybridization and/or wash at 6, 7, 8, 9, or 10° C. lower than the thermal melting point (Tₘ); low stringency conditions can utilize a hybridization and/or wash at 11, 12, 13, 14, 15, or 20° C. lower than the thermal melting point (Tₘ). Using the equation, hybridization and wash compositions, and desired Tₘ, those of ordinary skill will understand that variations in the stringency of hybridization and/or wash solutions are inherently described. If the desired degree of mismatching results in a Tₘ of less than 45° C. (aqueous solution) or 32° C. (formamide solution) it is preferred to increase the SSC concentration so that a higher temperature can be used. An extensive guide to the hybridization of nucleic acids is found in Tijssen, Laboratory Techniques in Biochemistry and Molecular Biology—Hybridization with Nucleic Acid Probes, Part I, Chapter 2 “Overview of principles of hybridization and the strategy of nucleic acid probe assays”, Elsevier, New York (1993); and Current Protocols in Molecular Biology, Chapter 2, Ausubel, et al., Eds., Greene Publishing and Wiley-Interscience, New York (1995).

To achieve an enhancement (e.g. over-expression), e.g. the number of copies of the corresponding genes is increased, or the promoter and regulation region or the ribosome binding site upstream of the structural gene is mutated. Expression cassettes which are incorporated upstream of the structural gene act in the same way. By inducible promoters, it is additionally possible to increase the expression in the course of fermentative pantothenic acid formation. The expression is likewise improved by measures to prolong the life of the m-RNA. Furthermore, the enzyme activity is also increased by preventing the degradation of the enzyme protein. The genes or gene constructs are either present here in plasmids with a varying number of copies, or are integrated and amplified in the chromosome. Alternatively, an over-expression of the genes in question can furthermore be achieved by changing the composition of the media and the culture procedure.

Instructions in this context can be found by the expert, inter alia, in Martin et al. (Bio/Technology 5, 137-146 (1987)), in Guerrero et al. (Gene 138, 35-41 (1994)), Tsuchiya and Morinaga (Bio/Technology 6, 428-430 (1988)), in Eikmanns et al. (Gene 102, 93-98 (1991)), in EP 0 472 869, in U.S. Pat. No. 4,601,893, in Schwarzer and Puhler (Bio/Technology 9, 84-87 (1991), in Reinscheid et al. (Applied and Environmental Microbiology 60, 126-132 (1994)), in LaBarre et al. (Journal of Bacteriology 175, 1001-1007 (1993)), in Patent Application WO 96/15246, in Malumbres et al. (Gene 134, 15-24 (1993)), in Japanese Laid-Open Specification JP-A-10-229891, in Jensen and Hammer (Biotechnology and Bioengineering 58, 191-195 (1998)) and in known textbooks of genetics and molecular biology.

By way of example, the zwa1 gene was over-expressed with the aid of plasmids.

Suitable plasmids are those which are replicated in Coryneform bacteria. Numerous known plasmid vectors, such as e.g. pZ1 (Menkel et al., Applied and Environmental Microbiology (1989), 64: 549-554), pEKEx1 (Eikmanns et al., Gene 102:93-98 (1991)), or pHS2-1 (Sonnen et al., Gene 107:69-74 (1991)) are based on the cryptic plasmids pHM1519, pBL1 or pGA1. Other plasmid vectors, such as e.g. those based on pCG4 (U.S. Pat. No. 4,489,160), or pNG2 (Serwold-Davis et al., FEMS Microbiology Letters 66, 119-124 (1990)), or pAG1 (U.S. Pat. No. 5,158,891), can be used in the same manner.

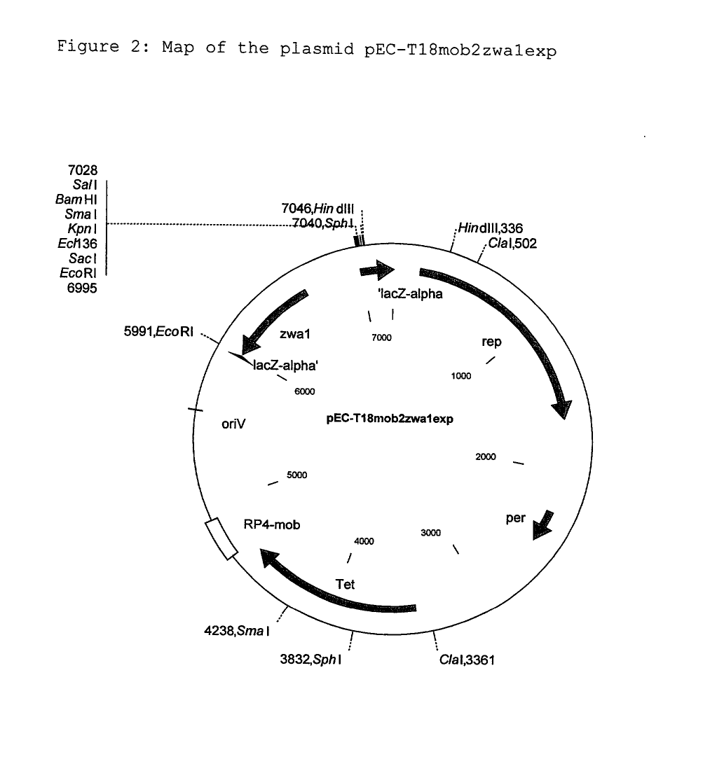

An example of a replicative plasmid vector is the plasmid vector pEC-T18mob2zwa1exp shown in FIG. 2.

Plasmid vectors which are moreover suitable are those with the aid of which the process of gene amplification by integration into the chromosome can be used, as has been described, for example, by Reinscheid et al. (Applied and Environmental Microbiology 60, 126-132 (1994)) for duplication or amplification of the hom-thrB operon. In this method, the complete gene is cloned in a plasmid vector which can replicate in a host (typically E. coli), but not in C. glutamicum.

Possible vectors are, for example, pSUP301 (Simon et al., Bio/Technology 1, 784-791 (1983)), pK18mob or pK19mob (Schafer et al., Gene 145, 69-73 (1994)), pGEM-T (Promega corporation, Madison, Wis., USA), pCR2.1-TOPO (Shuman (1994). Journal of Biological Chemistry 269:32678-84; U.S. Pat. No. 5,487,993), pCR®Blunt (Invitrogen, Groningen, Holland; Bernard et al., Journal of Molecular Biology, 234: 534-541 (1993)) or pEM1 (Schrumpf et al, 1991, Journal of Bacteriology 173:4510-4516). The plasmid vector which contains the gene to be amplified is then transferred into the desired strain of C. glutamicum by conjugation or transformation. The method of conjugation is described, for example, by Schafer et al. (Applied and Environmental Microbiology 60, 756-759 (1994)). Methods for transformation are described, for example, by Thierbach et al. (Applied Microbiology and Biotechnology 29, 356-362 (1988)), Dunican and Shivnan (Bio/Technology 7, 1067-1070 (1989)) and Tauch et al. (FEMS Microbiological Letters 123, 343-347 (1994)). After homologous recombination by means of a “cross over” event, the resulting strain contains at least two copies of the gene in question.

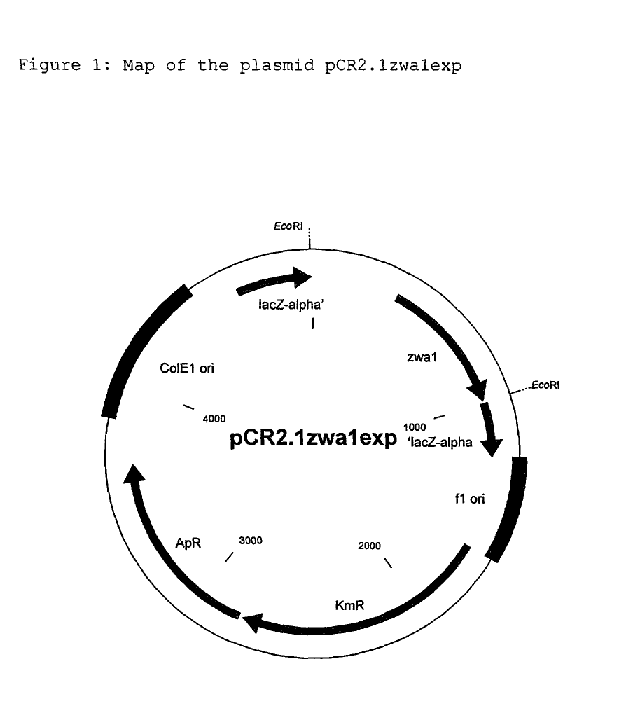

An example of an integration vector is the integration plasmid pCR2.1zwa1 exp shown in FIG. 1.

For production of pantothenic acid, it may additionally be advantageous for one or more further genes which code for enzymes of the pantothenic acid biosynthesis pathway or the keto-isovaleric acid biosynthesis pathway, in addition to the zwa1 gene, such as e.g.

the panB gene which codes for ketopantoate hydroxymethyltransferase (Sahm et al., Applied and Environmental Microbiology, 65, 1973-1979 (1999)), or

the panC gene which codes for pantothenate synthetase (Sahm et al., Applied and Environmental Microbiology, 65, 1973-1979 (1999)), or

the ilvD gene which codes for dihydroxy acid dehydratase

to be enhanced, in particular over-expressed.

In addition to over-expression of the zwa1 gene it may furthermore be advantageous for the production of pantothenic acid to eliminate undesirable side reactions (Nakayama: “Breeding of Amino Acid Producing Microorganisms”, in: Overproduction of Microbial Products, Krumphanzl, Sikyta, Vanek (eds.), Academic Press, London, UK, 1982).

The microorganisms prepared according to the invention can be cultured continuously or discontinuously in the batch process (batch culture) or in the fed batch (feed process) or repeated fed batch process (repetitive feed process) for the purpose of pantothenic acid production. A summary of known culture methods is described in the textbook by Chmiel (Bioprozesstechnik 1. Einführung in die Bioverfahrenstechnik [Bioprocess Technology 1. Introduction to Bioprocess Technology (Gustav Fischer Verlag, Stuttgart, 1991)) or in the textbook by Storhas (Bioreaktoren und periphere Einrichtungen [Bioreactors and Peripheral Equipment] (Vieweg Verlag, Braunschweig/Wiesbaden, 1994)).

The culture medium to be used must meet the requirements of the particular microorganisms in a suitable manner. Descriptions of culture media for various microorganisms are contained in the handbook “Manual of Methods for General Bacteriology” of the American Society for Bacteriology (Washington D.C., USA, 1981).

Sugars and carbohydrates, such as e.g. glucose, sucrose, lactose, fructose, maltose, molasses, starch and cellulose, oils and fats, such as e.g. soya oil, sunflower oil, groundnut oil and coconut fat, fatty acids, such as e.g. palmitic acid, stearic acid and linoleic acid, alcohols, such as e.g. glycerol and ethanol, and organic acids, such as e.g. acetic acid, can be used as the source of carbon. These substance can be used individually or as a mixture.

Organic nitrogen-containing compounds, such as peptones, yeast extract, meat extract, malt extract, corn steep liquor, soya bean flour and urea, or inorganic compounds, such as ammonium sulfate, ammonium chloride, ammonium phosphate, ammonium carbonate and ammonium nitrate, can be used as the source of nitrogen. The sources of nitrogen can be used individually or as a mixture.

Potassium dihydrogen phosphate or dipotassium hydrogen phosphate or the corresponding sodium-containing salts can be used as the source of phosphorus. The culture medium must furthermore comprise salts of metals, such as e.g. magnesium sulfate or iron sulfate, which are necessary for growth. Finally, essential growth substances, such as amino acids and vitamins, can be employed in addition to the abovementioned substances.

Precursors of pantothenic acid, such as aspartate, β-alanine, ketoisovalerate, ketopantoic acid or pantoic acid, and optionally salts thereof, can moreover be added to the culture medium to additionally increase the pantothenic acid production. The starting substances mentioned can be added to the culture in the form of a single batch, or can be fed in during the culture in a suitable manner.

Basic compounds, such as sodium hydroxide, potassium hydroxide, ammonia, or acid compounds, such as phosphoric acid or sulfuric acid, can be employed in a suitable manner to control the pH of the culture. Antifoams, such as e.g. fatty acid polyglycol esters, can be employed to control the development of foam. Suitable substances having a selective action, e.g. antibiotics, can be added to the medium to maintain the stability of plasmids. To maintain aerobic conditions, oxygen or oxygen-containing gas mixtures, such as e.g. air, are introduced into the culture. The temperature of the culture is usually 20° C. to 45° C., and preferably 25° C. to 40° C. Culturing is continued until a maximum of pantothenic acid has formed. This target is usually reached within 10 hours to 160 hours.

The concentration of pantothenic acid formed can be determined with known chemical (Velisek; Chromatographic Science 60, 515-560 (1992)) or microbiological methods, such as e.g. the Lactobacillus plantarum test (DIFCO MANUAL, 10^{th} Edition, p. 1100-1102; Mich., USA).

The following microorganism was deposited on 19th October 1999 as a pure culture at the Deutsche Sammlung fur Mikroorganismen und Zellkulturen (DSMZ=German Collection of Microorganisms and Cell Cultures, Braunschweig, Germany) in accordance with the Budapest Treaty:

Escherichia coli Top10′/pCR2.1zwa1exp as DSM13115.

The present invention is explained in more detail in the following with the aid of embodiment examples.

For this purpose, experiments were carried out with the isoleucine-requiring strain ATCC13032ΔilvA and the plasmid pND-D2. A pure culture of the strain ATCC13032ΔilvA has been deposited on Oct. 21, 1998 as DSM12455 at the Deutsche Sammlung für Mikroorganismen und Zellkulturen [German Collection of Microorganisms and Cell Cultures] in Braunschweig (Germany) in accordance with the Budapest Treaty. The plasmid pND-D2 containing the panD gene is described in Dusch et al. (Applied and Environmental Microbiology 65(4), 1530-1539 (1999)) and has also been deposited in the form of a pure culture of the strain Corynebacterium glutamicum ATCC13032/pND-D2 on Oct. 5, 1998 as DSM12438 at the Deutsche Sammlung für Mikroorganismen und Zellkulturen in Braunschweig [German Collection of Microorganisms and Cell Cultures] (Germany) in accordance with the Budapest Treaty.

Having generally described this invention, a further understanding can be obtained by reference to certain specific examples which are provided herein for purposes of illustration only, and are not intended to be limiting unless otherwise specified.

### EXAMPLES

### Example 1

Preparation of a genomic cosmid gene library from Corynebacterium glutamicum ATCC 13032

Chromosomal DNA from Corynebacterium glutamicum ATCC 13032 was isolated as described by Tauch et al. (1995, Plasmid 33:168-179) and partly cleaved with the restriction enzyme Sau3AI (Amersham Pharmacia, Freiburg, Germany, Product Description Sau3AI, Code no. 27-0913-02). The DNA fragments were dephosphorylated with shrimp alkaline phosphatase (Roche Molecular Biochemicals, Mannheim, Germany, Product Description SAP, Code no. 1758250). The DNA of the cosmid vector SuperCos1 (Wahl et al. (1987) Proceedings of the National Academy of Sciences, USA 84:2160-2164), obtained from Stratagene (La Jolla, USA, Product Description SuperCos1 Cosmid Vector Kit, Code no. 251301) was cleaved with the restriction enzyme XbaI (Amersham Pharmacia, Freiburg, Germany, Product Description XbaI, Code no. 27-0948-02) and likewise dephosphorylated with shrimp alkaline phosphatase. The cosmid DNA was then cleaved with the restriction enzyme BamHI (Amersham Pharmacia, Freiburg, Germany, Product Description BamHI, Code no. 27-0868-04).

The cosmid DNA treated in this manner was mixed with the treated ATCC 13032 DNA and the batch was treated with T4 DNA ligase (Amersham Pharmacia, Freiburg, Germany, Product Description T4-DNA-Ligase, Code no.27-0870-04). The ligation mixture was then packed in phages with the aid of Gigapack II XL Packing Extract (Stratagene, La Jolla, USA, Product Description Gigapack II XL Packing Extract, Code no. 200217). For infection of the E. coli strain NM554 (Raleigh et al. 1988, Nucleic Acid Research 16:1563-1575) the cells were taken up in 10 mM MgSO₄ and mixed with an aliquot of the phage suspension. The infection and titering of the cosmid library were carried out as described by Sambrook et al. (1989, Molecular Cloning: A Laboratory Manual, Cold Spring Harbor), the cells being plated out on LB agar (Lennox, 1955, Virology, 1:190) with 100 μg/ml ampicillin. After incubation overnight at 37° C., recombinant individual clones were selected.

### Example 2

Isolation and sequencing of the zwa1 gene

The cosmid DNA of an individual colony was isolated with the Qiaprep Spin Miniprep Kit (Product No. 27106, Qiagen, Hilden, Germany) in accordance with the manufacturer's instructions and partly cleaved with the restriction enzyme Sau3AI (Amersham Pharmacia, Freiburg, Germany, Product Description Sau3AI, Product No. 27-0913-02). The DNA fragments were dephosphorylated with shrimp alkaline phosphatase (Roche Molecular Biochemicals, Mannheim, Germany, Product Description SAP, Product No. 1758250). After separation by gel electrophoresis, the cosmid fragments in the size range of 1500 to 2000 base pairs were isolated with the QiaExII Gel Extraction Kit (Product No. 20021, Qiagen, Hilden, Germany).

The DNA of the sequencing vector pZero-1, obtained from Invitrogen (Groningen, The Netherlands, Product Description Zero Background Cloning Kit, Product No. K2500-01) was cleaved with the restriction enzyme BamHI (Amersham Pharmacia, Freiburg, Germany, Product Description BamHI, Product No. 27-0868-04). The ligation of the cosmid fragments in the sequencing vector pZero-1 was carried out as described by Sambrook et al. (1989, Molecular Cloning: A Laboratory Manual, Cold Spring Harbor), the DNA mixture being incubated overnight with T4 ligase (Pharmacia Biotech, Freiburg, Germany). This ligation mixture was then electroporated (Tauch et al. 1994, FEMS Microbiol Letters, 123:343-7) into the E. coli strain DH5αMCR (Grant, 1990, Proceedings of the National Academy of Sciences U.S.A., 87:4645-4649) and plated out on LB agar (Lennox, 1955, Virology, 1:190) with 50 Aμ/ml zeocin.

The plasmid preparation of the recombinant clones was carried out with the Biorobot 9600 (Product No. 900200, Qiagen, Hilden, Germany). The sequencing was carried out by the dideoxy chain termination method of Sanger et al. (1977, Proceedings of the National Academy of Sciences, USA, 74:5463-5467) with modifications according to Zimmermann et al. (1990, Nucleic Acids Research, 18:1067). The “RR dRhodamin Terminator Cycle Sequencing Kit” from PE Applied Biosystems(Product No. 403044, Weiterstadt, Germany) was used. The separation by gel electrophoresis and analysis of the sequencing reaction were carried out in a “Rotiphoresis NF Acrylamide/Bisacrylamide” Gel (29:1) (Product No. A124.1, Roth, Karlsruhe, Germany) with the “ABI Prism 377” sequencer from PE Applied Biosystems (Weiterstadt, Germany).

The raw sequence data obtained were then processed using the Staden program package (1986, Nucleic Acids Research, 14:217-231) version 97-0. The individual sequences of the pZero1 derivatives were assembled to a continuous contig. The computer-assisted coding region analyses were prepared with the XNIP program (Staden, 1986, Nucleic Acids Research, 14:217-231).

The resulting nucleotide sequence of the zwa1 gene is shown in SEQ ID NO 1. Analysis of the nucleotide sequence showed an open reading frame of 597 base pairs, which was called the zwa1 gene. The zwa1 gene codes for a polypeptide of 199 amino acids, which is shown in SEQ ID NO 2.

### Example 3

Preparation of an integration vector for over-expression of the zwa1 gene

From the strain ATCC 13032, chromosomal DNA was isolated by the method of Eikmanns et al. (Microbiology 140: 1817 -1828 (1994)). On the basis of the sequence of the zwa1 gene known for C. glutamicum from Example 2, the following oligonucleotides were chosen for the polymerase chain reaction:

| | |
|---|---|
| zwal-d1: | |
| 5′ TCA CA CCG ATG ATT CAG CC 3′ | (SEQ ID NO:3) |
| zwal-d2: | |
| 5′ AGA TTT AGC CGA CGA AAG CG 3′ | (SEQ ID NO:4) |

The primers shown were synthesized by MWG Biotech (Ebersberg, Germany) and the PCR reaction was carried out by the standard PCR method of Innis et al. (PCR protocols. A guide to methods and applications, 1990, Academic Press) with Pwo-Polymerase from Boehringer. With the aid of the polymerase chain reaction, a DNA fragment approx. 1.0 kb in size was isolated, this carrying the zwa1 gene.

The amplified DNA fragment was ligated with the TOPO TA Cloning Kit from Invitrogen Corporation (Carlsbad, Calif., USA; Catalogue Number K4500-01) in the vector pCR2.1-TOPO (Mead at al. (1991) Bio/Technology 9:657-663). The E. coli strain Top10F′ was then electroporated with the ligation batch (Hanahan, In: DNA cloning. A practical approach. Vol. I. IRL-Press, Oxford, Washington D.C., USA). Selection of plasmid-carrying cells was carried out by plating out the transformation batch on LB Agar (Sambrook et al., Molecular cloning: a laboratory manual. 2^{nd Ed. Cold Spring Harbor Laboratory Press, Cold Spring Harbor, N.Y.), which had been supplemented with} 25 mg/l kanamycin. Plasmid DNA was isolated from a transformant with the aid of the QIAprep Spin Miniprep Kit from Qiagen and checked by restriction with the restriction enzyme EcoRI and subsequent agarose gel electrophoresis (0.8%). The plasmid was called pCR2.1zwa1exp and is shown in FIG. 1.

### Example 4

Preparation of a replicative plasmid for expression of the zwa1 gene

4.1 Preparation of the shuttle vector pEC-T18mob2

The E. coli—C. glutamicum shuttle vector pEC-T18mob2 was constructed according to the prior art. The vector contains the replication region rep of the plasmid pGA1 including the replication effector per (U.S. Pat. No. 5,175,108; Nesvera et al., Journal of Bacteriology 179, 1525-1532 (1997)), the tetracycline resistance-imparting tetA(Z) gene of the plasmid pAG1 (U.S. Pat. No. 5,158,891; gene library entry at the National Center for Biotechnology Information (NCBI, Bethesda, Md., USA) with Accession Number AF121000), the replication region oriV of the plasmid pMB1 (Sutcliffe, Cold Spring Harbor Symposium on Quantitative Biology 43, 77-90 (1979)), the lacZα gene fragment including the lac promoter and a multiple cloning site (mcs) (Norrander et al. Gene 26, 101-106 (1983)) and the mob region of the plasmid RP4 (Simon et al.,(1983) Bio/Technology 1:784-791).

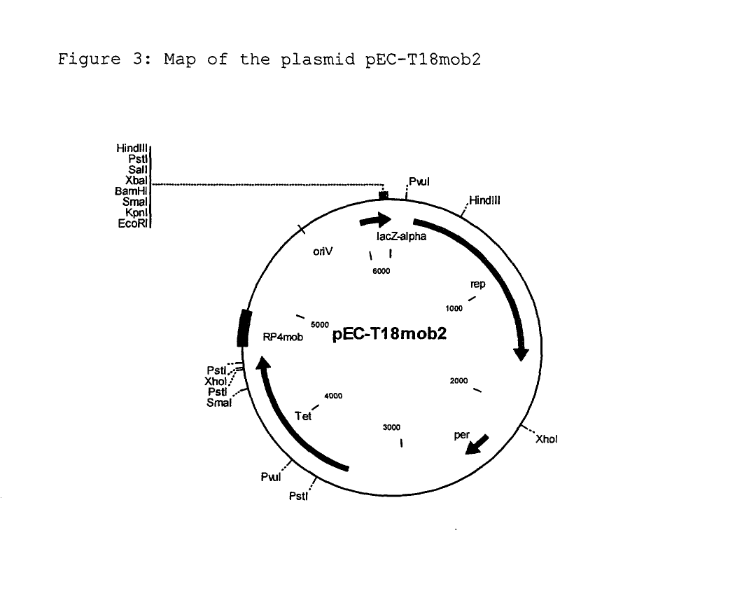

The vector constructed was transformed in the E. coli strain DH5α (Hanahan, In: DNA cloning. A Practical Approach, Vol. I, IRL-Press, Oxford, Washington D.C., USA). Selection for plasmid-carrying cells was made by plating out the transformation batch on LB agar (Sambrook et al., Molecular Cloning: A Laboratory Manual, 2^{nd} Ed., Cold Spring Harbor Laboratory Press, Cold Spring Harbor, N.Y.), which had been supplemented with 5 mg/l tetracycline. Plasmid DNA was isolated from a transformant with the aid of the QIAprep Spin Miniprep Kit from Qiagen and checked by restriction with the restriction enzymes EcoRI and HindIII and subsequent agarose gel electrophoresis (0.8%). The plasmid was called pEC-T18mob2 and is shown in FIG. 3.

A pure culture of the strain DH5α/pEC-T18mob2 was deposited on Jan. 20, 2000 at the Deutsche Sammlung fur Mikroorganismen und Zellkulturen (DSMZ=German Collection of Microorganisms and Cell Cultures, Braunschweig, Germany) as DSM 12344 in accordance with the Budapest Treaty.

4.2 Cloning of zwa1 in the shuttle vector pEC-T18mob2

The E. coli—C. glutamicum shuttle vector pEC-T18mob2 described in Example 4.1 was used as the vector. DNA of this plasmid was cleaved completely with the restriction enzyme EcoRI and then dephosphorylated with shrimp alkaline phosphatase (Roche Molecular Biochemicals, Mannheim, Germany, Product Description SAP, Product No. 1758250). For the insert, DNA of the plasmid pCR2.1zwa1exp was isolated from a transformant by the conventional method, digested with the restriction endonuclease EcoRI and ligated in the cleaved vector pEC-T18mob2. After the ligation, the batch was electroporated in the strain E. coli DH5αmcr. Selection was carried out on LB agar plates with 5 μg/ml tetracycline. Plasmid DNA from a transformant obtained in this way was isolated, cleaved with the restriction endonuclease EcoRI and the fragments were then checked by agarose gel electrophoresis. The plasmid constructed in this way was called pEC-T18mob2zwa1exp and is shown in FIG. 2.

### Example 5

Preparation of the strain ATCC13032ΔilvA/pND-D2, pEC-T18mob2zwa1exp

After electroporation (Tauch et. al., 1994, FEMS Microbiological Letters, 123:343-347) of the plasmid pND-D2 in the C. glutamicum strain ATCC13032ΔilvA and subsequent selection on LB agar (Lennox, 1955, Virology, 1:190-206), which had been supplemented with 25 μg/ml kanamycin, the strain ATCC13032ΔilvA/pND-D2 was obtained.

After electroporation of the plasmid pEC-T18mob2zwa1exp (Example 4) in the C. glutamicum strain ATCC13032ΔilvA/pND-D2 and subsequent selection on LB agar, which had been supplemented with 25 μg/ml kanamycin and 10 μg/ml tetracycline, the strain ATCC13032ΔilvA/pND-D2, pEC-T18mob2zwa1exp was obtained.

### Example 6

### Preparation of Pantothenic Acid

The formation of pantothenate by the C. glutamicum strains ATCC13032ΔilvA/pND-D2 and ATCC13032ΔilvA/pND-D2, pEC-T18mob2zwa1exp was tested in medium CGXII (Keilhauer et al., 1993, Journal of Bacteriology, 175:5595-5603; table 1), which had been supplemented with 25 μg/ml kanamycin, 2 mM isoleucine and in the case of the strain ATCC13032ΔilvA/pND-D2, pEC-T18mob2zwa1exp with additionally 10 μg/ml tetracycline.

This medium is called C. glutamicum test medium in the following. In each case 50 ml of freshly prepared C. glutamicum test medium were inoculated with a 16 hours old preculture of the same medium such that the optical density of the culture suspension (O.D.₅₈₀) at the start of incubation was 0.1. The cultures were incubated at 30° C. and 130 rpm. After incubation for 5 hours, IPTG (isopropyl β-D-thiogalactoside) was added in a final concentration of 1 mM. After incubation for 24 hours the optical density (O.D.₅₈₀) of the culture was determined and the cells were then removed by centrifugation at 5000 g for 10 minutes and the supernatant subjected to sterile filtration.

A Novaspec II photometer from Pharmacia (Freiburg, Germany) was employed at a measurement wavelength of 580 nm for determination of the optical density.

The D-pantothenate in the culture supernatant was quantified by means of Lactobacillus plantarum ATCC 8014 in accordance with the instructions in the handbook of DIFCO (DIFCO MANUAL, 10^{th} Edition, p. 1100-1102; Michigan, USA). The hemi-calcium salt of pantothenate from Sigma (Deisenhofen, Germany) was used for the calibration.

The result is shown in table 2.

TABLE 1

| Substance | Amount per liter | | Comments |
|---|---|---|---|
| (NH₄)₂ SO₂ | 20 | g | |
| Urea | 5 | g | |
| KH₂PO₄ | 1 | g | |
| K₂HPO₄ | 1 | g | |
| MgSO₄ * 7 H₂O | 0.25 | g | |
| MOPS | 42 | g | |
| CaCl₂ | 10 | mg | |
| FeSO₄ * 7 H₂O | 10 | mg | |
| MnSO₄ * H₂O | 10 | mg | |
| ZnSO₄ * 7 H₂O | 1 | mg | |
| CuSO₄ | 0.2 | mg | |
| NiCl₂ * 6 H₂O | 0.02 | mg | |
| Biotin | 0.5 | mg | |
| Glucose | 40 | g | autoclave |
| | | | separately |
| Protocatechuic acid | 0.03 | mg | sterile |
| | | | filtration |

TABLE 2

| | Cell density | Concentration |
|---|---|---|
| Strain | OD₅₈₀ | (ng/ml) |
| ATCC13032ΔilvA/pND-D2 | 11.5 | 8.4 |
| ATCC13032ΔilvA/pND-D2, | 12.8 | 11.7 |
| pEC-T18mob2zwalexp | | |

The base pair numbers stated are approximate values obtained in the context of reproducibility of measurements.

The abbreviations and designations used have the following meaning.

| | |
|---|---|
| Tet: | Resistance gene for tetracycline |
| KmR: | Resistance gene for kanamycin |
| ApR: | Resistance gene for ampicillin |
| ColEI ori | Replication origin ColE1 |
| oriV: | Plasmid-coded replication origin from E. coli |
| f1 ori: | Replication origin of phage f1 |
| RP4mob: | mob region for mobilizing the plasmid |
| rep: | Plasmid-coded replication origin from |
| | C. glutamicum plasmid pGA1 |
| per: | Gene for controlling the number of copies |
| | from pGA1 |
| lacZ-alpha: | lacZα gene fragment (N-terminus) of the |
| | β-galactosidase gene |
| ′ lacZ-alpha: | 3 ′ end of the lacZα gene fragment |
| lacZ-alpha ′: | 5 ′ end of the lacZα gene fragment |
| zwal: | zwal gene from C. glutamicum ATCC13032 |
| BamHI: | Cleavage site of the restriction enzyme |
| | BamHI |
| EcoRI: | Cleavage site of the restriction enzyme |
| | EcoRI |
| HindIII: | Cleavage site of the restriction enzyme |
| | HindIII |
| KpnI: | Cleavage site of the restriction enzyme KpnI |
| PstI: | Cleavage site of the restriction enzyme PstI |
| PvuI: | Cleavage site of the restriction enzyme PvuI |
| SalI: | Cleavage site of the restriction enzyme SalI |
| SacI: | Cleavage site of the restriction enzyme SacI |
| SmaI: | Cleavage site of the restriction enzyme SmaI |
| SphI: | Cleavage site of the restriction enzyme SphI |
| XbaI: | Cleavage site of the restriction enzyme XbaI |
| XhoI: | Cleavage site of the restriction enzyme XhoI |

Obviously, numerous modifications and variations on the present invention are possible in light of the above teachings. It is therefore to be understood that within the scope of the appended claims, the invention may he practiced otherwise than as specifically described herein.

What is claimed is:

1. A process for preparing D-pantothenic acid comprising
a. culturing a Coryneform bacteria comprising an enhanced zwa1 gene in a medium suitable for expression of the zwa1 gene; and
b. collecting the D-pantothenic acid produced.

2. The process of claim 1, wherein said zwa1 gene encodes a polypeptide comprising the amino acid sequence of SEQ ID NO:2.

3. The process of claim 1, wherein said zwa1 gene comprises the nucleotide sequence of SEQ ID NO:1.

4. The process of claim 1, wherein said zwa1 gene comprises a nucleotide sequence which hybridizes under stringent conditions to the nucleotide sequence of SEQ ID NO:1 and encodes a polypeptide having Zwa1 protein activity, wherein said stringent conditions comprise washing in 5×SSC at a temperature from 50 to 68° C.

5. The process of claim 1, wherein said enhanced zwa1 gene is overexpressed in said Coryneform bacteria.

6. The process of claim 1, wherein said Coryneform bacteria is Coryneform glutamicum.

7. The process of claim 1, wherein said Coryneform bacterium is selected from the group consisting of Coryneformbacterium acteoglutamicum, Coryneformbacterium acetoacidophilum, Coryneformbacterium thermoaminogenes, Brevibacterium flavum, Brevibacterium lactofermentum, and Brevibacterium divaricatum.

8. The process of claim 1, wherein said Coryneform bacterium further comprises at least one gene whose expression is enhanced, wherein said gene is selected from the group consisting of panB, panC, and ilvD.

9. The process of claim 1, wherein said Coryneform bacterium is Corynebacterium glutamicum DSM13115.

10. Corynebacterium glutamicum DSM13115.

11. A process for producing D-pantothenic acid comprising:
a. Transforming a Coryneform bacteria with a vector comprising a zwa1 gene, wherein said zwa1 gene is under the control of a promoter which allows the over-expression of said zwa1 gene;
b. culturing said transformed Coryneform bacteria in a medium suitable for expression of the zwa1 gene; and
c. collecting the D-pantothenic acid produced.

12. The process of claim 11, wherein said zwa1 gene encodes a polypeptide comprising the amino acid sequence of SEQ ID NO:2.

13. The process of claim 11, wherein said zwa1 gene comprises the nucleotide sequence of SEQ ID NO:1.

14. The process of claim 11, wherein said zwa1 gene comprises a nucleotide sequence which hybridizes under stringent conditions to the nucleotide sequence of SEQ ID NO:1 and encodes a polypeptide having phosphofructokinase activity, wherein said stringent conditions comprise washing in 5×SSC at a temperature from 50 to 68° C.

15. The process of claim 11, wherein said enhanced zwa1 gene is overexpressed in said Coryneform bacteria.

16. The process of claim 11, wherein said Coryneform bacteria is Coryneform glutamicum.

17. The process of claim 11, wherein said Coryneform bacterium is selected from the group consisting of Coryneformbacterium acteoglutamicum, Coryneformbacterium acetoacidophilum, Coryneformbacterium thermoaminogenes, Brevibacterium flavum, Brevibacterium lactofermentum, and Brevibacterium divaricatum.

18. The process of claim 11, wherein said Coryneform bacterium further comprises at least one gene whose expression is enhanced, wherein said gene is selected from the group consisting of panB, panC, and ilvD.

19. The process of claim 11, wherein said Coryneform bacterium is Corynebacterium glutamicum DSM13115.

20. A Coryneform bacteria comprising an enhanced zwa1 gene.

21. The Coryneform bacteria of claim 20, wherein said zwa1 gene encodes a polypeptide having Zwa1 protein activity.

22. The Coryneform bacteria of claim 21, wherein said Zwa1 protein comprises the amino acid sequence of SEQ ID NO:2.

23. The Coryneform bacteria of claim 20, wherein said zwa1 gene comprises the nucleotide sequence of SEQ ID NO:1.


SEQUENCE LISTING (4 sequences)

SEQ ID NO: 1 - DNA, 1260 bp, Corynebacterium glutamicum
  -10_signal (383)..(388)
ccgaaatattccaaatatgtaacataaatcacacccgatgattcaggcgggatgacctgcgacttcaaggtcgcaccaaagtcagattgatatagatttcgtaaataacgtgacacaatcgtgaccttcgggttaccgtgtatcccaggcaccgcaacagttcatctgcaagtccggctcatcgccaaaccctgtctggggtcggaagttgaacaacctccttggtgcaacagaactttaaaccacaaactcccgcattcatgtgggccatattgcagacagggacggggaaaccacccaccatcttttcacaaaagaaggcatggaggccaactccttggggtgaagccagacatccactggcagagcaactcctccgctctaacccgacagctaacctcgacggcgacaaatgagaggaaaacttttcatgggacgtcactccactaagactagctccgcgttcaccaagctcgcagcttccaccatcgctttcggtgctgctgcaaccatcatggctccttctgcatctgctgcacctgattccgactgggatcgcctcgcacagtgcgagtccggtggtaactgggcaatcaacaccggtaacggctaccacggtggtctgcagttctccgctagcacctgggctgcttacggcggccaggagttcgctacctacgcataccaggcaacccgtgagcagcagatcgctgttgcagagcgcaccttggctggtcagggctggggcgcatggcctgcttgctccgcttcccttggactgaactccgctccaacccagcgtgacctctccgctaccacctccaccccagagccagctgcagctgcaccagctgttgctgagtacaacgctcctgcagccaacatcgcagttggctccaccgacttgaacaccatcaagtccacctacggcgctgtcaccggcaccctcgctcagtacggcatcaccgttccagctgaggttgagtcttactacaacgctttcgtcggctaaatctagctgcactttttaaaagggagggaaccttaaacgggttccctccctttttgcatgccatttcacgacgcgccagtcatccttttgtgaattgggcaccaagatttcctgattttggccaccattttgccgaaaccttggtgccgaaagtacgcccagtagaaaaaccgcatgaaaaaagaggcaacaccgccgaaacgggttgcctcttttttaagtttcttagcggttgatccgggtgtacg

SEQ ID NO: 2 - protein, 199 aa, Corynebacterium glutamicum
MRGKLFMGRHSTKTSSAFTKLAASTIAFGAAATIMAPSASAAPDSDWDRLAQCESGGNWAINTGNGYHGGLQFSASTWAAYGGQEFATYAYQATREQQIAVAERTLAGQGWGAWPACSASLGLNSAPTQRDLSATTSTPEPAAAAPAVAEYNAPAANIAVGSTDLNTIKSTYGAVTGTLAQYGITVPAEVESYYNAFVG

SEQ ID NO: 3 - DNA, 19 bp, Corynebacterium glutamicum
tcacaccgatgattcaggc

SEQ ID NO: 4 - DNA, 20 bp, Corynebacterium glutamicum
agatttagccgacgaaagcg